(19) United States
(12) Patent Application Publication
(10) Pub. No.: US 2012/0130159 A1
(43) Pub. Date: May 24, 2012

(54) HOLDING SYSTEM FOR MEDICAL INSTRUMENTS

(76) Inventors: Omid Abri, Berlin (DE); Klaus-Martin Irion, Emmingen-Liptingen (DE); Stephan Schrader, Kleinmachnow (DE)

(21) Appl. No.: 13/304,130

(22) Filed: Nov. 23, 2011

(30) Foreign Application Priority Data
Nov. 24, 2010 (DE) 10 2010 052 219.8

(51) Int. Cl. A61B 1/04; F21V 21/00; G03B 17/56; F16M 13/00

(52) U.S. Cl. 600/102; 248/288.11

(57) ABSTRACT

A holding system for medical instruments with at least one bracket for fastening a medical instrument and at least one joint for positioning the one bracket and/or instrument. The holding system is configured to integrate in the bracket at least one of: light source, image recording unit, image display unit, control unit for the holding system, control unit for external components, sensor for distance, surface scanning and/or recording conditions of the environment, medical device for minimally invasive surgery, cooling unit, and/or wireless data communication unit. Accordingly, fewer or shorter feeder lines are required, gaining degrees of freedom of movement for the surgeon, reducing the possibility of stumbling in the operating room.

### CROSS-REFERENCE TO RELATED APPLICATIONS

The present application claims priority of German patent application No. 10 2010 052 219.8 filed on Nov. 24, 2010, the content of which is incorporated herein by reference.

### FIELD OF THE INVENTION

The present invention relates to a holding system for medical instruments with at least one bracket to which a medical instrument can be attached and with at least one joint for positioning the bracket and/or the medical instrument.

### BACKGROUND OF THE INVENTION

Holding systems of this type are often required in performing surgical procedures in order to hold medical instruments of a wide range of types, such as retractors, endoscopes or video cameras, in a particular position for a considerable period of time. Owing to the jointed configuration of the holding system, it becomes possible for the surgeon to precisely position the instrument held by the bracket and to stabilize the selected position of the holding system by blocking or fixing the joint or joints of the bracket.

A generically similar holding system is known, for example, from German patent application DE 195 21 060 A1. This known holding system consists of several brackets that are connected with one another by joints. A medical instrument is fastened or blocked on the distal end of the holding system. Feeder and supply lines are run outside the holding system and strongly restrict the operator's freedom of movement.

An additional generically similar holding system is described in German utility model DE 92 18 373 U1. This document too relates to a holding system with several brackets that are jointedly connected with one another. A medical instrument can be fastened on the distal end of the holding system. The feeder lines for individual apparatuses, such as connector cables, are grouped in a single supply hose and conducted along the length of the brackets, partly inside a bracket. This feeding of the supply hose inside the arm has the disadvantage that the sterilization requirements can only be met with considerable difficulty. In addition, as a result of the cable feeding, there is a considerable loss of power, which increases enormously, in particular, with additional interfaces and can result in significant losses of light when one light source is in use.

### SUMMARY OF THE INVENTION

It is consequently the object of the invention to provide a holding system for medical instruments of the aforementioned type, which reduces as far as possible the disadvantages cited above. At the same time, the cable feeders and supply lines are also intended to be improved as much as possible without restricting functionality.

This object is fulfilled by means of the characteristics of Patent claim 1. Advantageous refinements of the invention are the subject of the additional claims.

The inventive holding system for medical instruments, equipped with at least one bracket on which a medical instrument can be secured, and provided with at least one joint for positioning the at least one bracket and/or the medical instrument, is configured according to the invention in such a way that at least one functional structure, integrated into the bracket, contains at least one of the following units:
a) light source,
b) image recording unit,
c) image display unit,
d) control unit for controlling the holding system,
e) control unit for controlling external components,
f) sensor for scanning distance, scanning surfaces, and/or recording conditions in the environment,
g) medical apparatuses for use in minimally invasive surgery,
h) cooling unit,
i) wireless data communication unit.

As a result of this inventive configuration of the holding system, fewer or shorter feeder lines are required to the medical instrument that is to be held, a factor that is also associated with a greater degree of freedom of movement for the surgeon. Also, thanks to the reduction of the feeder lines, there are fewer risks of stumbling in the operating room, appreciably increasing safety in the operating room. The same applies as well when the inventive holding system is made available in treatment locations, for example in an ear-nose-throat or gynecological treatment facility. The invention is explained hereinafter using the example of an arrangement in an operating room, although this presentation is also applicable analogously to a treatment location.

The integration of several functional components directly into the holding system is also advantageous in making it possible to avoid additional equipment carts, which often obstruct movement during an operation. In this way the operator can directly control all or many of the important functions for a procedure and is not required to leave the operating table during the procedure, because the holding system is located in his or her immediate sphere of activity. Thus the inventive holding system replaces additional equipment carts in the operating room, at least in part. As a result of the advantageous integration of at least one component directly into the holding system, it becomes simpler to control the components required during a procedure. Thus, thanks to the invention, the regular disturbance caused by leaving the surgical area is no longer necessary. Thus the invention results in an increase in security in the operating room.

The invention makes it possible to avoid cable defects that occur from equipment carts often being rolled over cables that lay in their path. Thanks to the inventive holding system, feeder lines, cables and supply hoses extend directly from a bracket of the holding system, replacing cables that previously extended throughout the room, and thus can no longer restrict the operator's freedom of movement. In addition to freedom of movement gained by the inventive integration and the resulting routing of cables in the holding system, additional freedom of movement results from the fact that possible equipment carts can be dispensed with at least proportionately because the corresponding components are integrated directly into the holding system and thus are constantly in the proximity of the surgical area.

Because of cables lying around, there were previously very many cases of dysfunction caused by electrical and electromagnetic couplings. Said dysfunctions have been appreciably reduced by the inventive object. Combined and defined arrangement of the feeder lines inside the bracket reduces susceptibility to dysfunction to a demonstrable degree. In addition, feeder lines can be additionally insulated or cordoned off in the interior of the holding system and fed directly to the component in the holding system by a short route.

If the holding system has more than one bracket available, then said brackets can be connected by way of one or more joints so that the holding system can be moved in several degrees of freedom. The at least one component is advantageously integrated in the distal area of the holding system or else in the distal area of the bracket in order to avoid additional cable feeds or lines. The distal area of the holding system is the area to which the medical instrument can also be affixed. The proximal area of the holding system is the area situated away from the distal area that is, for example close to the ceiling, floor, or table mounting. In theory, the holding system can be mounted on the ceiling or on the operating table, or else can be secured or installed independently. The point of attachment of the holding system is thus typically set up in the proximal area.

It is advantageously proposed that a modular component system should be integrated into the bracket. Said system can consist of one or more components that are, in particular, integrated into the bracket, in such a way that each individual component in turn comprises one or more functional units according to the invention. In a preferred embodiment, two components, for example, are integrated into the bracket, so that one of the two components comprises a light source while the other component comprises the control unit and a data communication unit. Other combinations of the components or of the integrated units are of course possible in the modules in the context of the invention. As a result of the modular structure, it is possible without great complication, according to the invention, to rapidly adapt the holding system to the particular surgical procedure and to remove or replace the brackets with the corresponding components or integrated units in the modules, so that the necessary functional units are always directly situated in the vicinity of the surgical site. In addition, as a result of the bundled cables being fed in the bracket, possible mechanical, electrical and/or electromagnetic malfunction, such as constantly arise from cables freely lying about, can be prevented.

According to a preferred embodiment of the invention, it is proposed that a light source should be integrated into the module of the bracket. The advantage of an integrated light source consists in the fact that a light source outside the holding system with a power line that is integrated into the holding system requires an additional interface, which results in significant light losses. This additional interface is not necessary with an integrated light source, and thus light losses are also reduced. In addition, the transmission pathways for the light are typically reduced, leading again to a better light yield. An LED light source is advantageously used. Consequently a more compact structure is possible than with the use of other light sources such as a xenon light source. Moreover, in using an LED light source, less heat is generated and there is a high capacity density. The high degree of effectiveness of LED is thus especially advantageous in converting or integrating into an inventive holding system.

It is proposed in addition that the image recording unit, which is integrated into the bracket, should advantageously contain an endoscopic video camera. The endoscopic video camera has at least one image recorder at its disposal and also, in particular, is advantageously removably integrated in the distal area of the bracket. The mechanical interface between the holding system and the medical instrument is preferably formed by the coupling “video camera/medical instrument.” It is an advantage of this configuration that the video signal supply line also comes from the bracket and thus no additional disturbing cables, extending laterally, are required. It has also proved especially advantageous here for the video camera to be integrated in such a way that its field of operation can continue to operate when in integrated state. Another advantage with this embodiment is that it requires no separate means of operation for distant components, for example on an equipment cart. Thus it is not necessary to leave the surgical area. With this embodiment, the risks of stumbling are markedly reduced in the operating room. In addition it regularly occurred in the past that feeder lines to the image recording unit lay in the way and were crossed by equipment carts and the like being pushed through and thus were damaged. In this case the image signal was no longer available to the operator and the operation had to be interrupted. Thanks to the inventive integration of the image recording unit and the feeder lines in the holding system, these disadvantages are eradicated. In addition, the system's propensity to malfunction is diminished by a reduction of optical or electrical cables and supply lines lying or hanging in the vicinity.

In a preferred embodiment of the invention, in the distal area of the bracket of the holding system there is an electromechanical interface, by which a video endoscope can be coupled with an integrated image recorder. The endoscope here is preferably a cost-effective disposable endoscope. Both the electrical power supply and/or electronic image signal and the mechanical coupling of the video endoscope occur by way of the electromechanical interface. It is also possible to power the light by way of the interface. It has proved especially advantageous to configure the electromechanical interface in such a way that the mechanical input for an endoscope is configured in tubular shape either as tubes open on both sides or as tubes closed below, in the manner of a reaction tube. It is also preferable here to associate several tubular interfaces with one series, improving the handling and electronic connection. It is precisely through the use of tubes closed on one side, the lower side, that it becomes possible to apply a disinfectant solution to free the interposed endoscope from infectious impurities.

If an image display unit, in particular an image projection unit, is advantageously integrated in the bracket, then patient-specific data, among other things, or an endoscopic image can be projected into the operator's field of vision, preferably in the direction of the patient surface, in particular in the surgical area. Preferably a DLP (digital light processing) projector is used as image projector and thus the image is generated onto the patient in the surgeon's field of vision.

In an additional preferred embodiment of the invention, a video camera as well as the corresponding related signal processing is integrated into a bracket, precisely in connection with an image display unit. The advantage of this embodiment, besides the clear reduction of cables, is the positioning of the video camera close to the surgical site, where said video camera can provide the operator, without losses in quality, with the necessary image information, for instance in the form of an image, through the image display unit that is integrated into the holding system. The system's susceptibility to malfunction against electrical and electromagnetic effects is reduced, incidents of stumbling in the operating room are eliminated and there is reduced risk of sudden loss of image because of cables lying around that easily caused people to stumble and possibly to damage them. All these positive effects result in an increase in safety in the operating room and/or during a procedure.

In an additional embodiment, control units are integrated in one or more brackets of the holding system. This refers both to control units that directly guide the holding system itself and also to control units that can direct functions or components outside the holding system. The control of other surgical devices directly by the holding system has proved especially effective. A further advantage of this embodiment is that owing to the inventive additional control of these components, the other control can serve as fallback position also in case of the failure of a control unit. In addition, the operator thus can also operate components that are spatially separated from the surgical area, without losing sight of the patient. The field of operation of the control unit is preferably applied directly in the vicinity of, or on the distal end of, the holding system so that direct operation is possible without leaving the surgical area.

In another preferred embodiment of the invention, a sensor is integrated into the bracket for distance surface scanning and/or recording of surrounding conditions. Examples of such a sensor include a time-of-flight camera or a triangulation sensor. Ultrasound sensors, IR sensors or video-based sensors are also recommended for recording the surroundings, according to the invention. As a result, the operating table along with the patient and instruments can be surveyed continuously and in terms of points, lines or surfaces. Consequently it becomes possible, according to the invention, in addition to the position of the instruments, to record the patient status and position and if necessary to track the holding system or instrument and/or patient in the position. Also, according to the invention, the operating table movements as well as the position, in particular of medical instruments connected with the distal end of the holding system, can be recorded for surgical navigation purposes. The sensors can be used, in addition, to record gestures by the operator and to interpret them as control signals, in particular for the holding system or other connected apparatuses. By recording the patient situation, it is also possible in particular to track the data and images of the projector of the patient status. Spatial recording of the patient as well as of the medical instruments and their position to one another provide surgical navigation support and/or make it possible to superimpose or match patient or navigation data generated pre-operatively or inter-operatively. By recording table movements in relation to the holding system, collisions by the patient or operating table with the holding system can be avoided. By recording the operator's gestures, the holding system can be powered without contact and thus in sterile conditions. Other surgical components can also be used thereby, without the need to leave the operating table and thus the patient and without complex, cumbersome cabling.

According to the invention, one or more of these position sensors are integrated in the distal area of the holding system. Thus, even in the event of a spatial rotation of the distal bracket, with the endoscope attached to it, in relation to the securing of the holding system or patient or operating table as a point of reference, the endoscopic image can be adjusted by software technology and thus can be displayed in correct position, with the help of the starting signal of the position sensors.

It is also proposed with the invention that the integrated video camera or the integrated electronic image recorder should be positioned rotatably in the distal area of the holding system. Then, if position sensors are integrated in the holding system and the holding system is spatially rotated, this is recorded by the position sensors. A signal is thereupon forwarded on to the power drive of the rotatable video camera or to the power drive of the electronic image recorder by means of a control unit, and adjustment of the image is controlled mechanically, in particular electro-mechanically, and thus not by means of software technology. Horizontal compensation can advantageously be achieved with these embodiments. The advantage of this configuration is that the endoscopic image is constantly held upright even in case of movements of the bracket and thereby a very secure handling is ensured even with a malfunctioning holding system.

In addition, with an additional preferred embodiment of the invention it is proposed that Bowden cables should be integrated in the bracket or brackets. The one end of the Bowden cables is connected with the video camera or with the image sensor, while the other end is fastened to a proximally situated bracket, in such a way that this bracket secured to the Bowden cable has a fixed spatial position, at least in one degree of freedom. The Bowden cable is secured in such a way that the video camera or the image sensor remains constantly upright in case of a spatial rotation of the distal bracket in which the video camera or electronic image sensor is rotatably mounted. For this purpose a transmission mechanism is provided in particular, which transmits the movement of the Bowden cable in terms of specific position to the bracket of the video camera or of the image sensor in transmission-correct manner.

According to an embodiment of the invention it is proposed that medical equipment (for example, flushing/suction pumps, HF cutting or coagulation devices, laser cutting or coagulation devices, ultrasound lithotripters) for use in minimally invasive surgery should be integrated directly in the bracket. In this embodiment it is also advantageous that the cables are integrated in the holding system and emerge centrally from the holding system and that no laterally extending cables should exist that would unnecessarily restrict the operator's freedom of movement. Unnecessary equipment carts, which previously have made equipment available and often obstructed traffic, are thus eliminated. In addition, pushing the equipment carts always involved considerable use of force and it was not rare that the cables leading to the individual appliances on the equipment cart were run over or lay in the way, preventing the motion of the equipment car. It was also not unusual that, upon pushing the equipment cart, the length of the feeder lines and cables was not taken into account and with cables that were too short the corresponding medical equipment would fall off the equipment carts, becoming damaged in the process. It is also advantageous with the integrated arrangement that additional pathways for operating components located outside the surgical area are avoided. The integration of additional functions in the holding system results in an increase in safety in the operating room. Thus the integration into the holding system is designed in such a way that the equipment is removed from the brackets or inserted into them and thus in simple manner can be subjected to maintenance, repairs or adjustment to the particular type of operation and thus to the operating requirements.

In addition, it is proposed with the invention that at least one cooling unit should be integrated in the holding system. Because there are sometimes a good number of components or functional units integrated in the holding system, there can be considerable waste heat to and in the holding system. It has proved useful, in addition, to integrate a cooling unit in the holding system so that heat developing there can be efficiently diverted from components integrated in the holding system. Without a cooling unit, harmful heating could develop quickly and in some cases could cause failure of integrated components. During an operation this would result in a forced stoppage, in some cases endangering the patient's life.

In one embodiment of the invention it is proposed to integrate, in particular, a wireless data communication unit in the holding system. Cable-less data exchange thereby becomes possible with devices located inside or, in particular, outside the surgical area. As a result, the number of feeder lines that could restrict the operator's freedom of movement is further reduced.

In an additional embodiment of the invention, the holding system comprises a power drive. Electromagnetic, piezoelectric, pneumatic and/or hydraulic power drives have proved especially effective for this purpose. An advantage of this embodiment is that defined positions can automatically be targeted. This is especially important when components are integrated into the holding system and said system thus comprises an enormous weight. “Operation by hand” of such a heavy holding system is possible only with considerable difficulty. Even a holding system with many integrated components can thus be positioned without problems by means of an appropriate power drive. With a corresponding power drive, it is also possible to configure the holding system without weight restrictions and thereby to make it accessible to simple positioning by grasping and pushing. In addition to the aforementioned power drives, compensation for weight is also possible by means of mechanical components such as for instance by counterweights.

It is further proposed according to the invention that the holding system should comprise a hollow structure. It is proposed preferably that both the at least one bracket and also the joints should comprise a pass-through hollow structure. As a result of the hollow structure of the holding system, the feeder lines and the necessary cables can be fed directly through the holding system. Thus no further cables lie in the operator's path. The susceptibility to interruptions is strongly reduced. By bundling the feeder lines in the holding system, the electromagnetic tolerance (EMT) in the operating room is improved outside the holding system and interruptions during an operation are reduced. This is achieved, first, by reducing the cable and feeder line lengths as well as by insulating the hollow structure by means of the metal of the holding system that surrounds it in the manner of a Faraday cage, as well as by additional EMT actions such as torsion of cables, additional insulations, groundings and the like.

According to an additional embodiment of the invention it is proposed that internal feeder lines and power outlet contacts, in particular for HF surgical devices, for motor-operated systems as well as for video cameras, should be integrated in the holding system or in the bracket. Thus additional cables can be avoided and central feeding of the cable is ensured without restricting the operator. This too serves to improve integration of the various cables or power lines, such as in the form of a central data or control bus or a central energy supply or centralized media supply as well as to enhance electromagnetic tolerance.

In an additional embodiment of the invention, a blower is integrated in the proximal area of the holding system. In combination with the hollow structure over the entire or essential part of the length of the holding system, air can be suctioned from the distal end with the help of the blower and can be diverted through the hollow structure to the proximal end. Active cooling of the holding system thereby becomes possible. The more components or units are integrated in the holding system, the more necessary becomes the integration of a cooler unit or of a blower in the holding system because failure of a component integrated in the bracket caused by overheating can lead to life-threatening situations. In addition to providing a blower, it has also proved effective to furnish a fluid-form pump, which advances fluid-form cooling agents, in particular distilled water, as a coolant in a fluid feeder system and thereby ensures sufficient cooling. This fluid feeder system extends in particular essentially over the entire length of the holding system. With comparative cooling capacity, the feeder lines have a relatively small diameter and thus occupy only a small portion of the hollow structure, so that sufficient room remains for other feeder lines or cables. This fluid feeder system is preferably configured in the manner of a simple motor vehicle cooling system.

In another embodiment of the invention, several passive heat conducting elements, in particular, are positioned inside the hollow structure. Thus the heat of the light source, for example, can advantageously be diverted inside the hollow structure toward the proximal direction. Embodiments of these heat conducting elements are, for instance, metallic rods or else heat pipes that are integrated inside the hollow structure of the bracket. Flex wires such as of copper are also possible as heat conducting elements in the jointed portions. At the proximally situated brackets the heat is preferably transmitted in surface form to the sheath elements of the bracket and diverted from there to the environment. Alternatively, parts of the heat conducting elements themselves form parts of the sheath elements. This embodiment of the invention has the advantage that overheating of the system is to a great extent avoided.

In addition, it is proposed with the invention that optical, magnetic and/or mechanical encoding elements should be integrated in individual and preferably in all joints in order to be able to record the position of the bracket. By means of the measured value encoder, it is possible to determine the present status or position of the particular bracket and thus of the holding system or of the medical instrument that is mounted on the distal end of the holding system. This facilitates the determination of the position of a possible navigation to a great extent. Linear coders are preferably used. Thus it is possible to use coders that comprise incremental, countable or else absolute measurement incorporations as line patterns, magnetization or contacts. The magnetic field modulation can be evaluated in the case of a permanent magnetization by means of AMR, GMR, Hall effect sensors or inductive sensors. The advantage of this configuration is that with the help of the output signal of the coder, a holding system equipped with it can perform reproducible movements. If an absolute value indicator is used according to the invention, that is if a clear signal pattern is associated with each position, then it is possible that the holding system after switching off can move back into a defined output position or reference position.

In one embodiment of the invention, with the help of integrated sensor technology and of corresponding encoder elements it is thus possible to adopt preset positions. Owing to the spatial recording of the operation environment and of the patient position, it is thereby possible to avoid collisions.

According to the invention, it is also proposed to integrate a control monitor in the holding system. If this monitor is advantageously incorporated distally in the bracket, said monitor can easily be observed and the displayed information can easily be recorded. In addition, on configuring the monitor as a touch screen, an input can occur and at the same time a targeted control of the holding system with its components thus becomes possible. An additional glance at a separate monitor, for instance on the wall of the operating room, that is at a distance from the surgical area, is therefore not necessary. The positioning of the holding system and a simultaneous image control, for example, are also possible in the viewing direction according to the invention.

This invention relates to a holding system that, in addition to its holding and positioning function as known in the art, also constitutes a functional platform, and several functional units are integrated in the holding system. In addition, the feeder lines are fed directly in the holding system in order to achieve an additional simplification. Owing to this configuration of the inventive holding system, a very universal means for retaining medical instruments is provided, into which, in addition to the holding function, a number of other functions of medical apparatuses are integrated that usually are performed by devices mounted on equipment carts in the operating room. The result is a holding system that is very functional and proves to be very secure with respect to operational safety in the operating room. It also proves to be very easy to operate.

Additional characteristics and advantages of the invention can be seen from the appended drawings, in which embodiments of an inventive holding system for medical instruments are illustrated by way of example. The invention is not restricted to these embodiments.

### BRIEF DESCRIPTION OF THE DRAWINGS

FIG. 1 shows an overview of an inventive holding system.

FIG. 2 shows a simplified view of the inventive holding system.

FIG. 3 shows a portion of the distal end of the holding system illustrated in FIG. 2.

FIG. 4 shows a portion of a distal end of an additional embodiment of the inventive holding system.

### DETAILED DESCRIPTION OF THE INVENTION

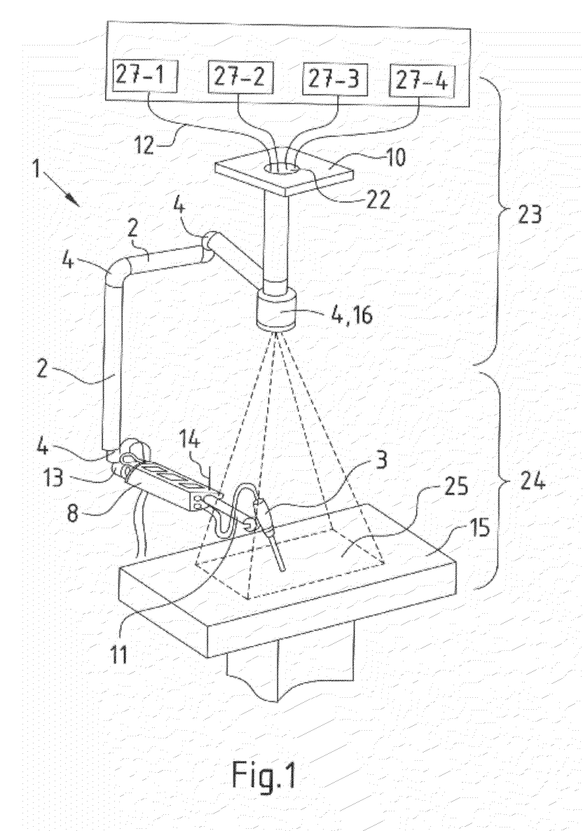

FIG. 1 shows an inventive holding system for medical instruments.

This holding system 1 consists essentially of several brackets 2, such that the individual brackets 2 are pivotably connected by joints 4 with one another so that they can be released and blocked in relation to one another.

Holding systems 1 of this type are often required in performing surgical procedures in order to hold medical instruments 3 of many kinds, such as retractors, video cameras, microscopes or endoscopes, in a particular position for extended periods. Owing to the jointed configuration of the holding system 1 it is possible for the surgeon to position the medical instrument 3 in the desired position and to fix the selected position of the holding system 1 by blocking the joint 4 or joints 4. In addition to endoscopic surgery, holding systems 1 of this type are also used in open surgery.

In the area of the proximal end 23, the holding system 1 can be secured, for instance to the ceiling, by a fixing unit 10 configured as a level plate. The holding system 1 can also be fastened by a fixer unit 10 on the operating table 15, on a wall of the operating room or on something else.

On the distal end 24 the bracket 2 comprises an instrument insertion point 11 for receiving the medical instrument 3 that is to be positioned by the holding system 1. As can be seen from FIG. 1, the instrument insertion point 11 is located in immediate proximity to the operating table 15.

In the embodiment according to FIG. 1, the feeder lines 12 of the various medical devices 27-1, 27-2, 27-31, 27-4, which in this embodiment are positioned proximally in the ceiling area, in particular in a subceiling, are fed through a hollow structure 22 extending through the entire holding system 1 or the bracket 2. According to the invention, modules with various functional units corresponding to one or more inserted medical devices are positioned so as to be integrated in one of the brackets 2 of the holding system 1.

According to the invention, an image display unit in the form of an image projection unit 16 is integrated into a joint 4 of a bracket 2. Patient-specific data or information, also in the form of image information, can thus be projected in the operator's field of vision and here directly into the surgical area 25 onto the operating table 15.

On the distal end 24 of the holding system 1, a cable connector 13 for cable hook-ups can be seen as well as an operating console with control unit 8 for controlling the holding system 1 or the components or else external components. The operating console with control unit 8, according to the invention, has input and display elements as well as an integrated light source 6. The inventive cable connector 13 makes possible a non-complicated, rapid connection of the holding system 1 with other medical devices that are necessary for the operation but are not integrated in the holding system 1. In addition to a cable connection 13, additional or other components can be integrated in the bracket 2 of the holding system 1.

Control units 8 can be configured in such a way that wireless data communication is possible via the transmitting and reception antennas 14, for example with various surgical devices or another operating room. The radio transmission can occur in the form of a Bluetooth connection, an infrared communication or the like. Here reference number 14 refers to an antenna that serves for radio transmission. The antenna 14 can of course be integrated in the control unit 8 and is thus not visible from the outside.

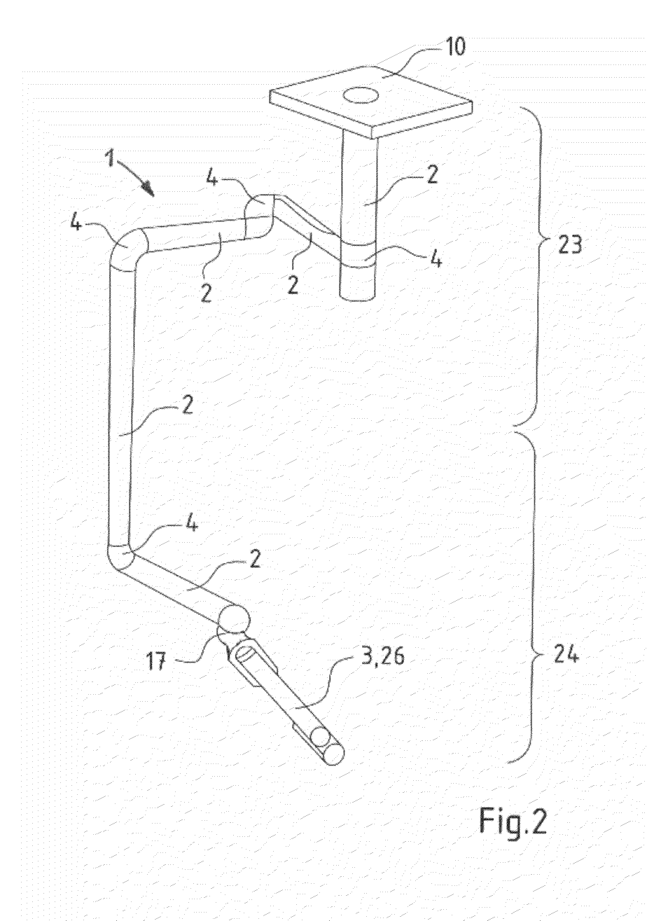

The holding system from FIG. 1 is shown in simplified form in FIG. 2. Here too, a fixing unit 10 can be recognized with which the proximal end 23 of the holding system 1 can be secured on the ceiling.

The holding system 1 has several brackets 2 available, which are movably connected with one another by joints 4. An electromechanical interface 17 is provided on the distal end 24 of the holding system 1. A video endoscope 3;26 with integrated image recorder is mechanically as well as electrically and data-technically connected with the holding system 1 via this interface. This video endoscope 26, according to the invention, is a disposable, reasonably priced endoscope.

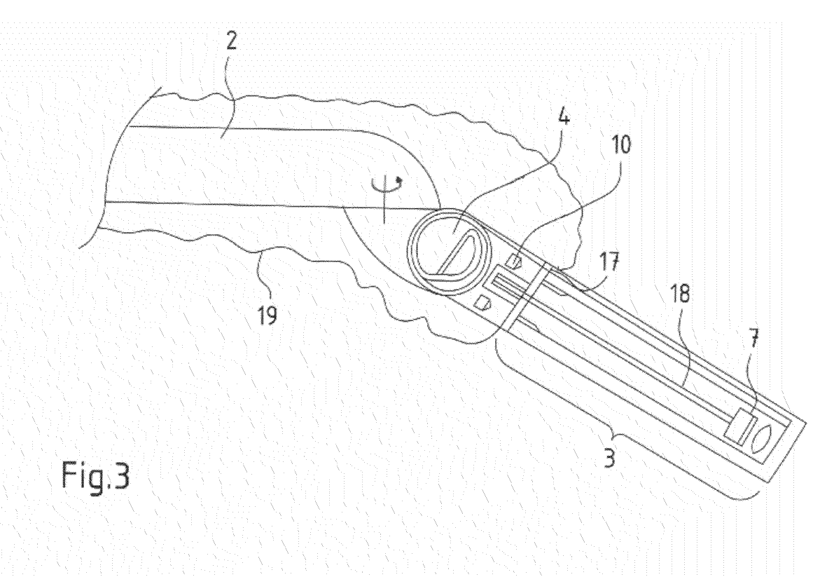

The distal end 24 of the holding system 1 from FIG. 2 is shown in greater detail in FIG. 3. The video endoscope is directly connected via an interface 17 with the bracket 2. The video endoscope 3;26 has at its disposal an image recording unit 7 configured as an integrated image receiver. The supply of electricity for the image recording unit 7, which is located in the video endoscope 3;26, occurs through a conductor plate 18 with proximal-end plug-in connection. The image recording unit 7 is located directly in the medical video endoscope 26 and is connected by the interface 17 with the bracket 2 via a conductor plate 18. The light source, which is configured as LED in this embodiment, is located in the bracket 2.

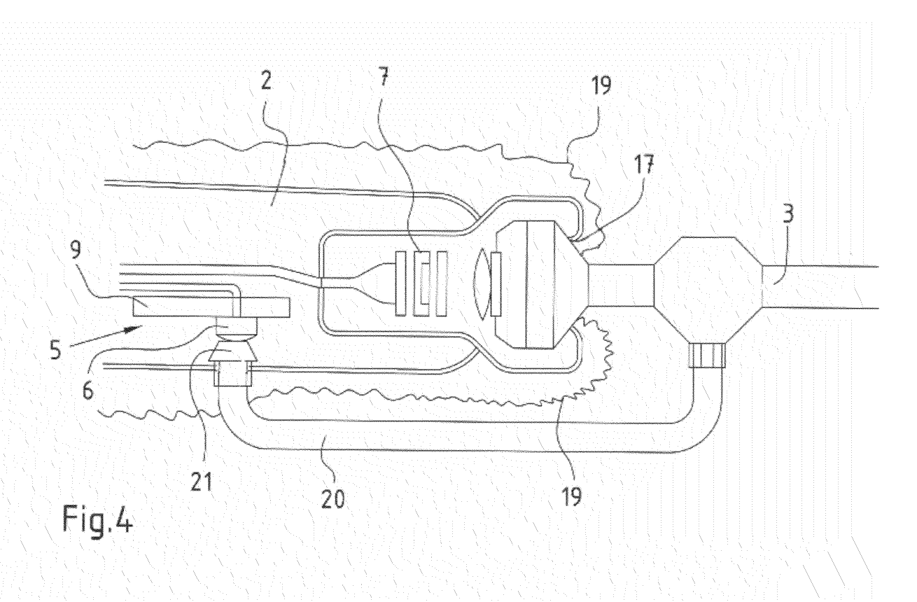

In the embodiments in FIGS. 3 and 4, the illustrated brackets 2 each have available a sterile covering 19, which extends to the distal end at the interface 17 between the bracket 2 and the medical instrument 3. The sterile covering 19 is used in order to ensure the sterility, in particular, of the holding system 1 in the operating room.

The distal end 24 of an additional embodiment of an inventive holding system 1 is illustrated in FIG. 4. The bracket 2 is equipped on its distal end 24 with a mechanical interface 17. This interface 17 connects the bracket 2 with the medical instrument 3, which in this embodiment constitutes an endoscope. In this case it is an interface 17 between the bracket 2 and the endoscope 3. Also integrated in the interface 17 is an image recording unit 7 with various lenses, CCD and evaluation switching circuits.

In the embodiment from FIG. 4, an LED semiconductor light source 6 is integrated in the bracket 2. This light source 6 with associated powering is positioned on the cooling body 9 and together with it forms a module 5. The cooling body 9 made up of plate-shaped heads ensures sufficient temperature equalization of the light source 6 and prevents overheating of the light source 6 and bracket 2 and/or of the functional units or modules integrated therein.

As can be seen in FIG. 4, in this embodiment the bracket 2 contains a light junction 21, which is connected with the medical instrument 3, an endoscope, via a light conductor.

Other or additional modules 6 can of course also be integrated in a bracket 2.

1. A holding system for medical instruments, with at least one bracket on which a medical instrument can be fastened and with at least one joint (4) for positioning the at least one bracket and/or the medical instrument,
characterized in that in the bracket at least one module is integrated, which contains at least one of the following functional units:
a) light source,
b) image recording unit,
c) image display unit,
d) control unit for controlling the holding system,
e) control unit for controlling external components,
f) sensor for distance, surface scanning and/or recording of environmental conditions,
g) medical devices for use in minimally invasive surgery,
h) cooling unit,
i) wireless data communication unit.

2. The holding system according to claim 1, wherein the light source comprises an LED as light source.

3. The holding system according to claim 1, wherein the image recording unit contains an endoscopic video camera.

4. The holding system according to claim 1, wherein the sensor for distance or surface scanning is a time of flight (TOF) video camera, a triangulation sensor, an ultrasound sensor and/or an IR sensor.

5. The holding system according to claim 1, wherein the bracket contains a power drive.

6. The holding system according to claim 5, wherein the bracket can be displaced by means of electromagnetic, piezoelectric, pneumatic and/or hydraulic drives.

7. The holding system according to claim 1, wherein the holding system comprises a hollow structure.

8. The holding system according to claim 1, wherein in the proximal area of the holding system a blower or a fluid pump is positioned that draws in air or another fluid from the distal side via the hollow structure of the holding system and conveys it toward the proximal side.

9. The holding system according to claim 7, wherein at least one passive heating element is positioned inside the hollow structure and diverts heat from the distal to the proximal side.

10. The holding system according to claim 9, wherein at least one passive heating element in the bracket is configured as a heat pipe.

11. The holding system according to claim 1, wherein one or more optical, magnetic and/or mechanical encoder elements are positioned in or on the joint to record the position of the bracket or brackets (2).

12. The holding system according to claim 11, wherein at least one actuator element is positioned in the joint with encoder element to position the bracket.

13. The holding system according to claim 1, wherein an electromagnetic interface for electronic linkage and mechanical insertion of an endoscope,.in particular a video endoscope, is provided in the distal area of the bracket of the holding system, such that in particular the mechanical insertion element of the electromechanical interface is of tubular-shaped configuration.

14. The holding system according to claim 2, wherein the image recording unit contains an endoscopic video camera.

15. The holding system according to claim 8, wherein at least one passive heating element is positioned inside the hollow structure and diverts heat from the distal to the proximal side.
